(19) United States
(12) Patent Application Publication
(10) Pub. No.: US 2024/0233942 A9
(43) Pub. Date: Jul. 11, 2024

(54) INCREASING A TRAINING DATA VOLUME FOR IMPROVING A PREDICTION ACCURACY OF AN AI-BASED IOL DETERMINATION

(71) Applicant: Carl Zeiss Meditec AG, Jena (DE)

(72) Inventors: Hendrik Burwinkel, Munich (DE); Michael Trost, Oberkochen (DE); Nicolas Bensaid, Oberkochen (DE); Stefan Saur, Oberkochen (DE)

(73) Assignee: Carl Zeiss Meditec AG, Jena (DE)

(21) Appl. No.: 18/492,985

(22) Filed: Oct. 24, 2023

(30) Foreign Application Priority Data
Oct. 25, 2022 (DE) 102022128198.1

(51) Int. Cl. G16H 50/20

(52) U.S. Cl. CPC G16H 50/20

(57) ABSTRACT

A computer-implemented method for increasing a training data volume for a machine learning system for determining an initial refractive power value for an intraocular lens to be inserted is described. The method includes measuring a group of ophthalmological biometry data of a patient and determining an initial refractive power value for the intraocular lens to be inserted by a trained machine learning system. The measured ophthalmological biometry data and a postoperative target refraction value are used as input data for the trained machine learning system. The method also includes measuring a postoperative refractive results value, assigning the postoperative refractive results value to the measured ophthalmological biometry data of the patient, and determining an importance indicator value for the new training data record.

### CROSS REFERENCE TO RELATED APPLICATIONS

This U.S. patent application is a continuation of, and claims priority under 35 U.S.C. § 119(d) to German Patent Application DE 10 2022 128 198.1, filed on Oct. 25, 2022. The disclosure of this prior application is considered part of the disclosure of this application and is hereby incorporated by reference in its entirety.

### TECHNICAL FIELD

The disclosure relates to a computer-implemented method for determining a refractive results value during a surgical cataract operation and to a method for determining a refractive power for an intraocular lens to be inserted during a surgical cataract operation. The disclosure further relates to corresponding systems and associated computer program products.

The disclosure also relates to an increase in a training data volume for a machine learning system, and more precisely to a computer-implemented method for increasing a training data volume for a machine learning system for determining an initial refractive power value for an intraocular lens to be inserted or for determining a postoperative refractive results value of an intraocular lens to be inserted. The disclosure further relates to a corresponding system and to corresponding computer program products.

### BACKGROUND

Machine learning systems (ML systems) are currently used in many scientific surroundings, and also in practical daily life. Machine learning systems “learn” their conversion of input data to output data using example data, which are known as training data. The volume and quality of the training data available for a learning process or for a training are decisive in this case with regards to the response of the ML system to unknown data. It is normally true that a larger volume of training data creates better predictions.

However, individual sectors frequently only have limited training data volumes available. This frequently also applies to ophthalmology, in which artificial intraocular lenses replace the crystalline lens in the eye within the scope of cataract operations. Trained machine learning systems for prediction of refractive power values for intraocular lenses are increasingly being supplied to surgical centers and used there. Renewed training with an augmented set of training data does not always take place and especially does not occur on a regular basis. In particular, additional training data from other clinics are used too infrequently, with the result that the available training data volume regularly remains rather subcritical.

Previous approaches of addressing this problem generally consisted of patient data being exchanged between clinics or the manufacturer of such learning systems. However, as a rule, this was found to be complicated from an organizational point of view and susceptible to errors because, inter alia, the data from different clinics are frequently stored in different formats.

Moreover, a clinic has difficulties estimating how valuable collected data are with regards to updating a machine learning system, and whether an update justifies the outlay connected therewith.

Consequently, there is a need to address the inadequacies of the existing solutions, in particular to propose a method and corresponding systems to this end, which allow a continual increase in the volume of training data.

### SUMMARY

According to a first aspect of the present disclosure, a computer-implemented method for increasing a training data volume for a machine learning system for determining an initial refractive power value for an intraocular lens to be inserted is presented. In this case, the method includes measuring a group of ophthalmological biometry data of a patient and determining an initial refractive power value for the intraocular lens to be inserted, by means of a trained machine learning system which was trained by means of an initial training data volume consisting of tuples of previously measured ophthalmological biometry data of patients, associated postoperative refractive results values, generated by means of a previously inserted intraocular lens, and associated initial refractive power values of the previously inserted intraocular lens as ground truth data for determining a corresponding machine learning model. Here, the measured ophthalmological biometry data and a postoperative target refraction value can be used as input data for the trained machine learning system.

Moreover, the method includes measuring a postoperative refractive results value, assigning the postoperative refractive results value to the measured ophthalmological biometry data of the patient in order to form a new training data record, and determining an importance indicator value for the new training data record, with the importance indicator value being indicative of a distance value of the new training data record in comparison with the initial training data volume.

The determination of the importance indicator value may additionally include determining at least one element from the following group: (i) a specification regarding a nominal improvement in the future refractive power value predictions by the machine learning system; and (ii) a specification regarding a refractive error that could have been avoided if the new training data record were known prior to the determination of the initial refractive power value.

According to a second aspect of the present disclosure, an associated system for increasing a training data volume for a machine learning system for determining a refractive power value for an intraocular lens to be inserted is presented. To this end, the system may comprise a processor and a memory which is operatively connected to the processor and which stores program code elements which, when executed, cause the processor to measure a group of ophthalmological biometry data of a patient and determine an initial refractive power value for the intraocular lens to be inserted, by means of a trained machine learning system. The latter may have been trained using an initial training data volume, the initial training data volume consisting of: (i) tuples of previously measured ophthalmological biometry data of patients; (ii) associated postoperative refractive results values, generated by means of a previously inserted intraocular lens; and (iii) associated initial refractive power values of the previously inserted intraocular lens as ground truth data for determining a corresponding machine learning model. Here, the measured ophthalmological biometry data and a postoperative target refraction value can be used as input data for the trained machine learning system.

The processor can also be caused to measure a postoperative refractive results value, assign the postoperative refractive results value to the measured ophthalmological biometry data of the patient in order to form a new training data record, and determine an importance indicator value for the new training data record, with the importance indicator value being indicative of a distance value of the new training data record in comparison with the initial training data volume.

In this case, the determination of the importance indicator value may additionally include determining at least one element from the following group: (i) a specification regarding a nominal improvement in the future refractive power value predictions by the machine learning system; and (ii) a specification regarding a refractive error that could have been avoided if the new training data record were known prior to the determination of the initial refractive power value.

According to a third aspect of the present disclosure, a computer-implemented method for increasing a training data volume for a machine learning system for determining a postoperative refractive results value of an intraocular lens to be inserted is presented. In this case, the method includes measuring a group of ophthalmological biometry data of a patient, and determining a postoperative refractive results value of the intraocular lens to be inserted, by means of a trained machine learning system. The trained machine learning system may have been trained using an initial training data volume consisting of: (i) tuples of previously measured ophthalmological biometry data of patients; (ii) associated initial refractive power values of a previously inserted intraocular lens; and (iii) associated postoperative refractive results values, generated by means of the previously inserted intraocular lens, as ground truth data for determining a corresponding machine learning model. In this case, the measured ophthalmological biometry data and an initial refractive power value of the inserted intraocular lens can be used as input data for the trained machine learning system.

The method may also include measuring the postoperative refractive results value, assigning the postoperative refractive results value to the measured ophthalmological biometry data of the patient in order to form a new training data record, and determining an importance indicator value for the new training data record, with the importance indicator value being indicative of a distance value of the new training data record in comparison with the initial training data volume.

According to a fourth aspect of the present disclosure, an associated system for increasing a training data volume for a machine learning system for determining a postoperative refractive results value of an intraocular lens to be inserted is presented. To this end, the system may comprise a processor and a memory which is operatively connected to the processor and which stores program code elements which, when executed, cause the processor to measure a group of ophthalmological biometry data of a patient and determine a postoperative refractive results value of the intraocular lens to be inserted, by means of a trained machine learning system. This trained machine learning system may have been trained with (i) an initial training data volume consisting of tuples of previously measured ophthalmological biometry data of patients, (ii) associated initial refractive power values of a previously inserted intraocular lens, and (iii) associated postoperative refractive results values, generated by means of the previously inserted intraocular lens, as ground truth data for determining a corresponding machine learning model, wherein the measured ophthalmological biometry data and an initial refractive power value of the inserted intraocular lens are used as input data for the trained machine learning system.

The processor can also be caused to measure the postoperative refractive results value, assign the postoperative refractive results value to the measured ophthalmological biometry data of the patient in order to form a new training data record, and determine an importance indicator value for the new training data record, with the importance indicator value being indicative of a distance value of the new training data record in comparison with the initial training data volume.

The proposed computer-implemented method for increasing a training data volume for a machine learning system for determining an initial refractive power value for an intraocular lens to be inserted and the method according to the second aspect have a plurality of advantages and technical effects, which also apply accordingly to the associated systems:

The proposed method can ensure, especially in the context of cataract operations, a continual increase in the volume of training data used for the retraining of the underlying machine learning system with its machine learning model. This field of medicine, in particular, frequently has only small volumes of training data available, and so the predictions of the utilized ML systems in individual areas—i.e., individual indications—can only be made with a low prediction precision (corresponding to a low percentage prediction probability for a prediction value).

By assessing a new training data record in the context of the previously available training data—for example expressed by the importance indicator value—there is ongoing motivation for the users to supply new training data from newly added operations. This applies even more since the provision of training data generally means a significant outlay for the user. These feedback data which can be provided to the clinics may also consist of highlighting which prediction errors can be avoided in future. As a result, the user receives direct feedback as to how valuable the collected data are for improving the machine learning system and is motivated to update particularly important data.

The importance indicator value can thus be used to form a closed process, and this creates an incentive to increase the training data volume. It is possible to immediately identify the positive contribution the additional training data record can make to the already existing training data volume. Thus, the advantages of social media systems are used in this case and implemented in a new technical concept in order to be able to continually provide new data and hence improve ML systems/ML models to clinics for upcoming cataract operations.

As a result, the method proposed here can thus ensure that a surgeon, prior to their cataract operation, can always work with machine learning systems which are up to date as a result of renewed training with continually supplied training data.

In principle, this way of obtaining additional training data can also be transferred to other applications, be they medical or else non-medical.

Further exemplary implementations are presented below, which can have validity both in association with the methods and in association with the corresponding systems.

According to a further developed implementation of the system, the determination of the specification regarding a nominal improvement in the future refractive power predictions by the machine learning system may include temporarily adding the new training data record to the initial training data and determining a new prediction accuracy of the machine learning model. In this way, direct feedback becomes visible to the contributor who makes the additional training data record available. To determine the prediction accuracy, it is possible to resort to standardized input values for the machine learning system which were defined in advance, and the probability of a correctness of the created prediction can be determined. This can be implemented once with the new training data record and once without the new training data record. The specification about the nominal improvement can be derived from a comparison of the respective probability values.

According to an additionally advantageous implementation, the method may include increasing the importance indicator value if there are no training data within a previously defined radius around the new training data. The nominal value of the increase of the importance indicator value can be determined using the mathematically determined distance from the closest set of training data or a number of closest sets of training data, the number being determined in advance. The greater the distance, the higher the importance indicator value may turn out to be as well. A simple linear dependence would already suffice. However, it is also possible to apply more complicated distance determination methods for the multi-dimensional space of training data tuples.

According to an interesting implementation, the method may additionally include displaying, on a graphical unit, the importance indicator value and/or the specification about a nominal improvement and/or the specification about the refractive error. This may also contribute to increasing the motivation for supplying further training data.

According to an additional implementation of the method, provision can additionally be made of transmitting the measured postoperative refractive results value to a training data memory. This can ensure that no additionally collected training data are lost. It is also possible—where necessary—to normalize the new training data at this opportunity. Additionally, it is also possible here to determine whether the additional training data record “fits to the other training data records”. In this way, it is possible to eliminate measurement outliers or data otherwise not fitting to the training data record, which would have a deterioration of the prediction quality of the machine learning system as a consequence.

According to an augmented implementation, the method may additionally include renewed training—in particular retraining—the machine learning system using the initial training data and the new training data record or new training data records. This continually improves the prediction accuracy of the machine learning system.

According to a further advantageous implementation of the method, the importance indicator value can be determined after collecting an amount of training data in the form of postoperative refractive results values for given ophthalmological biometry data and inserted intraocular lenses, the amount being determined in advance. This can prevent excessive updating of the machine learning model and hence placing increased demands on additionally required computing power (due to the possibly excessive retraining). The frequency of determining the importance indicator value can also be defined on the basis of the average distance of the newly added training data records from older trainer data records.

According to an implementation of the method, the importance indicator value for a selected type of an intraocular lens can be increased by a predefined factor. In this case, the factor can be increased depending on an amount of training data for the selected type of intraocular lens in comparison with the overall amount of training data. A comparison with a number of different types of intraocular lenses may also be used.

In this case, it is also possible to consider that the factor is increased if the amount of training data for the selected type of intraocular lens is less than a threshold value determined in advance. Consequently, there are a number of options for influencing the probability indicator value and flexibly designing the proposed method. Further influencing variables for the probability indicator value are not precluded.

According to another implementation of the disclosure, the importance indicator value can be determined after a number—for example 10, 100, 1000 patients—of postoperative refractive results values for one type of inserted intraocular lenses, the number being determined in advance. This can also be used to save computing resources and measurement outliers can be eliminated by the automatic averaging.

According to an additional, advantageous implementation, the method may also include determining a number of measurements of the postoperative refractive results value by one entity, and transferring the number of measurements by the entity in comparison with the number of comparable measurements by other entities. For example, the entity can be a clinic performing such operations/measurements. Thus, the new training data records can initially be buffer stored before they are transferred to the central unit storing the overall volume of training data. Each clinic can independently decide whether they wish to transfer each individual new training data record or initially carry out buffer storing.

Furthermore, implementations can relate to one or more computer program products able to be accessed from a computer-usable or computer-readable medium that comprises program code for use by, or in conjunction with, a computer or other instruction processing systems. In the context of this description, a computer-usable or computer-readable medium can be any device that is suitable for storing, communicating, transferring, or transporting the program code.

### DESCRIPTION OF DRAWINGS

It should be pointed out that exemplary implementations of the disclosure may be described with reference to different implementation categories. In particular, some exemplary implementations are described with reference to a method, whereas other exemplary implementations may be described in the context of corresponding devices. Regardless of this, it is possible for a person skilled in the art to identify and to combine possible combinations of the features of the method and also possible combinations of features with the corresponding system from the description above and below—if not specified otherwise—even if these belong to different claim categories.

Aspects already described above and additional aspects of the present disclosure are evident inter alia from the exemplary implementations described and from the additional further specific implementations described with reference to the figures.

Preferred exemplary implementations of the present disclosure are described by way of example and with reference to the following figures:

FIG. 1 depicts a flowchart-like representation of an exemplary implementation of the computer-implemented method according to the disclosure for increasing a training data volume for a machine learning system for determining an initial refractive power value for an intraocular lens to be inserted.

FIG. 2 shows an eye with potential parameter measurement values.

FIG. 3 depicts a flowchart-like representation of an exemplary implementation for a second computer-implemented method for increasing a training data volume for a machine learning system for determining a postoperative refractive results value of an intraocular lens to be inserted.

FIG. 4 shows an exemplary cycle of training the machine learning system, using the training data volume, and increasing the training data volume.

FIG. 5 shows an exemplary interaction of a plurality of users with the machine learning system.

FIG. 6 shows an exemplary implementation of the system for increasing a training data volume for a machine learning system for determining a refractive power value for an intraocular lens to be inserted.

FIG. 7 shows an exemplary implementation of another system for increasing the training data volume for a machine learning system for determining a postoperative refractive results value.

FIGS. 8a and 8b illustrate exemplary implementations of a computer system which can interact with the systems according to FIG. 6 or FIG. 7.

FIGS. 9a and 9b explain what training data vectors are used for the two differently trained machine learning systems from FIGS. 8a and 8b.

FIG. 10 shows an exemplary implementation of a computer system suitable for use together with the already presented systems.

### DETAILED DESCRIPTION

In the context of this description, conventions, terms and/or expressions should be understood as follows:

The term “machine learning system” describes a system which creates prediction values (predictions) on the basis of input data. In this case, a machine learning system has not been programmed procedurally but “learns” its behavior on the basis of training with training data. In so doing, the machine learning system is provided with input data and desired output data as so-called “ground truth data”. Machine learning systems for the context specified here are typically based on neural networks. These can be implemented both as a software construct and in hardware. In addition to prediction data, machine learning systems are also able to provide an associated quality statement, for example in the form of “the predicted value is correct with a probability of x %”.

In this context, the difference between a machine learning system and a machine learning model should also be highlighted. The machine learning system represents the fundamental architecture of the learning system while the machine learning model is formed during the course of the training with training data.

The term “training data volume” may in this case describe the data volume used as a basis for training the ML system to form the machine learning model. The quality of the created learning model in this case depends directly on the quality and also on the volume of the available training data. In principle, it is true that a greater volume of data has a positive effect on the prediction accuracy and quality (without neglecting boundary regions such as over fitting, etc., in this case).

The term “new training data record” in this case describes a completely new data record of input data and actual output data measured in reality, which is to say the ground truth data. These may have the form of a tuple with elements of the measured ophthalmological biometry data of patients, the associated postoperative refractive results values of a previously inserted intraocular lens and the associated initial refractive power values of the previously inserted intraocular lens as ground truth data.

The term “intraocular lens” describes an artificial lens in this case, the latter being inserted into an eye by a surgeon, to replace the crystalline lens of an eye. A cataract operation is a typical application.

The term “refractive power value” describes the optical refractive power of a lens, for example measured in diopter.

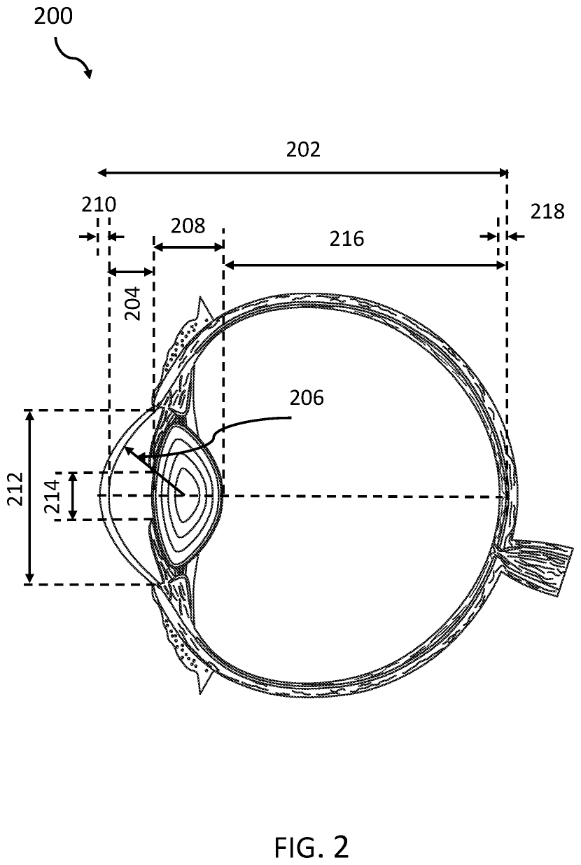

In the context of this disclosure, the term “ophthalmological biometry data” may predominantly describe the following variables of an eye: axial length (AL), anterior chamber depth (ACD) and the lens thickness of an eye. Further measurement values of the eye—e.g., as described in the context of FIG. 2—may additionally be available.

The term “postoperative target refraction value” may describe the effective refractive power value intended to be achieved following the insertion of the intraocular lens in the eye of the patient. It may differ from the postoperative refractive results value.

The term “postoperative refractive results value” may describe the effective refractive power value actually achieved following the insertion of the intraocular lens in the eye of the patient. This value—as mentioned previously—depends in particular on the actual position of the intraocular lens in the eye, into which the intraocular lens ultimately grows in the eye.

The term “importance indicator value” in this case describes, as it were, the value of a new training data record in the context of the already available training data. In principle, it may be true that the importance indicator value associated with regions in the multi-dimensional space of training data in which there was no or only little training data available previously, but which are now filled by a new training data record, turns out to be higher than the importance indicator value in regions where there already is a relatively large amount of training data.

A detailed description of the figures is given below. It is understood in this case that all of the details and instructions in the figures are illustrated schematically. Depicted initially is a flowchart-like representation of an exemplary implementation of the computer-implemented method according to the disclosure for increasing a training data volume for a machine learning system for determining an initial refractive power value for an intraocular lens to be inserted. Further exemplary implementations, or exemplary implementations for the corresponding system, are described below.

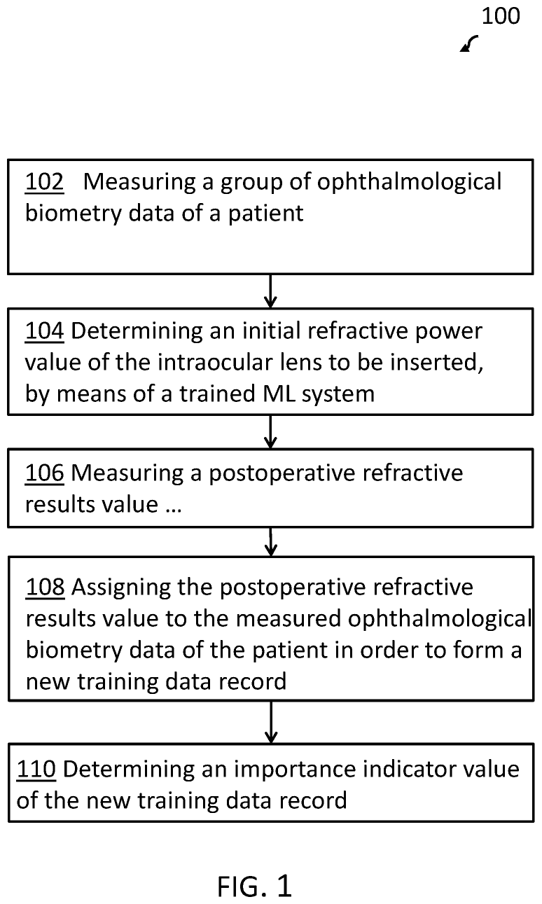

FIG. 1 depicts a flowchart-like representation of a preferred exemplary implementation of the computer-implemented method for increasing a training data volume for a machine learning system for determining an initial refractive power value for an intraocular lens to be inserted. The method includes measuring 102 a group of ophthalmological biometry data of a patient, and determining 104 an initial refractive power value for the intraocular lens to be inserted, by means of a trained machine learning system. In this case, the ML system was trained by means of an initial training data volume consisting of tuples of previously measured ophthalmological biometry data of patients, associated postoperative refractive results values of a previously inserted intraocular lens, and associated initial refractive power values of the previously inserted intraocular lens as ground truth data for determining a corresponding machine learning model. Each set of the training data volume can be represented by a vector or tuple. In this case, the measured ophthalmological biometry data and a postoperative target refraction value are used as input data for the trained machine learning system.

The method 100 additionally includes measuring 106 a postoperative refractive results value—e.g., by the clinic where the operation is performed—and assigning 108 the postoperative refractive results value to the measured ophthalmological biometry data of the patient—e.g., by way of a patient identifier—in order to form a new training data record.

Moreover, the method 100 includes determining 110 an importance indicator value for the new training data record, with the importance indicator value being indicative of a distance value of the new training data record in comparison with the initial training data volume. Known mathematical methods can be applied to determine the distance value. Since every training data record can be represented by a vector, it is possible to use known methods for determining distances between vectors (e.g., Euclidean distance).

In this case, the determination of the importance indicator value includes determining at least one element from the following group: (i) a specification regarding a nominal improvement in the future refractive power value predictions by the machine learning system; and (ii) a specification regarding a refractive error that could have been avoided if the new training data record were known prior to the determination of the initial refractive power value.

FIG. 2 illustrates an eye 200 with various biometric parameters of the eye. In

particular, the following parameters are illustrated: axial length 202 (AL); anterior chamber depth 204 (ACD); keratometry value 206 (K, radius); refractive power of the lens; lens thickness 208 (LT); central cornea thickness 210 (CCT); white-to-white distance 212 (WTW); pupil size 214 (PS); posterior chamber depth 216 (PCD); and retina thickness 218 (RT).

In a manner similar to FIG. 1, it is also possible to take the ML system-based determination of the postoperative refractive result value (instead of the initial refractive power value) as a basis.

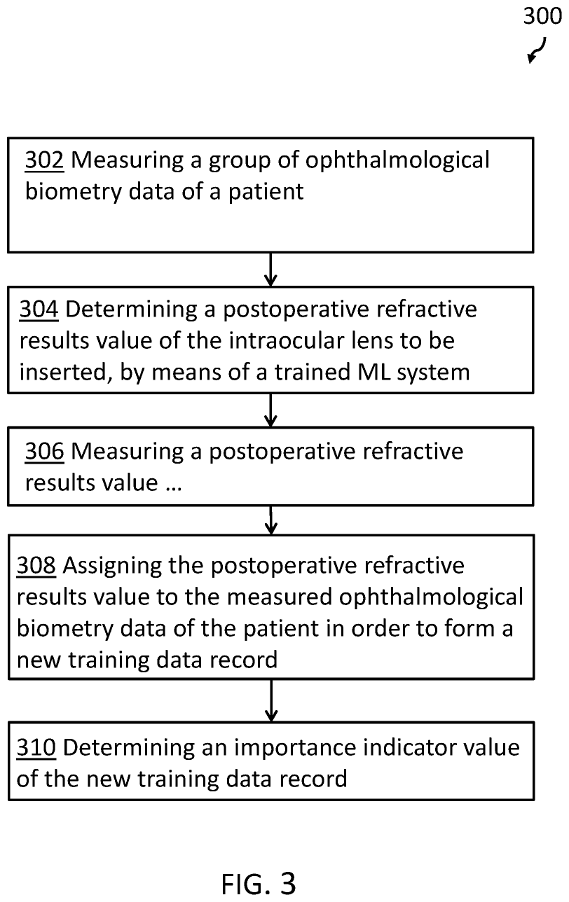

FIG. 3 shows a computer-implemented method for increasing a training data volume for a machine learning system for determining a postoperative refractive results value of an intraocular lens to be inserted. Thus, the method 300 accordingly includes measuring 302 a group of ophthalmological biometry data of a patient, and determining 304 a postoperative refractive results value of the intraocular lens to be inserted, by means of a trained machine learning system.

In this case, the machine learning system was trained by means of an initial training data volume consisting of tuples of previously measured ophthalmological biometry data of patients, associated initial refractive power values of a previously inserted intraocular lens, and associated postoperative refractive results values of the previously inserted intraocular lens as ground truth data for determining a corresponding machine learning model. Moreover, the measured ophthalmological biometry data and an initial refractive power value of the inserted intraocular lens are used as input data for the trained machine learning system.

Further, the method 300 includes measuring 306 the postoperative refractive results value, assigning 308 the postoperative refractive results value to the measured ophthalmological biometry data of the patient in order to form a new training data record, and determining 310 an importance indicator value for the new training data record, with the importance indicator value being indicative of a distance value of the new training data record in comparison with the initial training data volume.

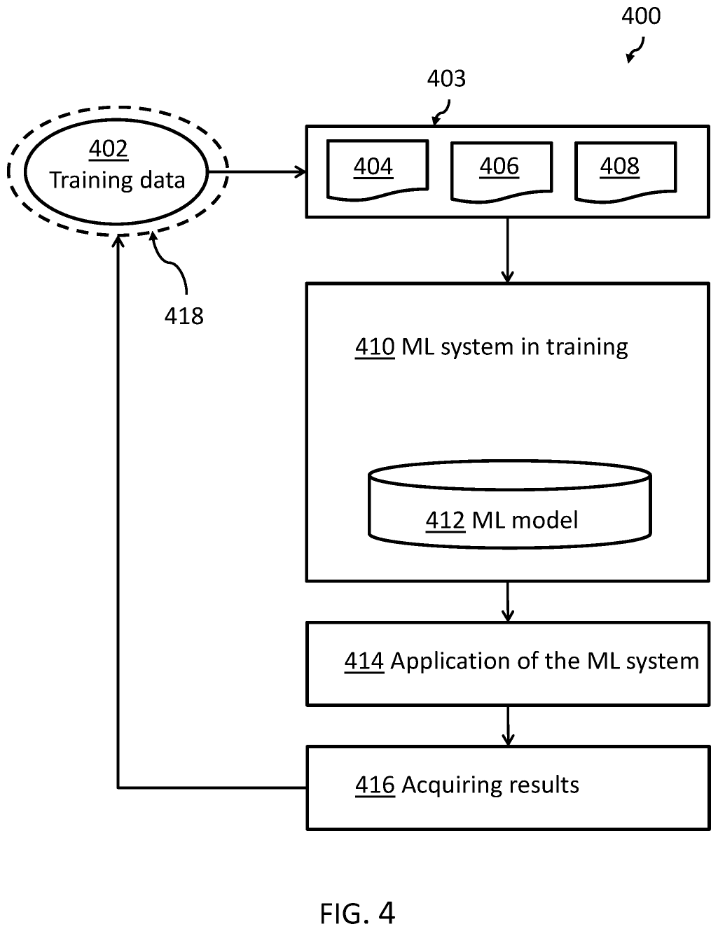

FIG. 4 shows an exemplary cycle 400 of training the machine learning system, using 414 the ML system, and increasing the training data volume 402. At the outset, this training data volume 402 is only available as initial training data volume, which consists of a plurality of sets 403 of training data. For example, these may include the following data in the form of tuples or vectors: measured ophthalmological biometry data 404 of patients, associated postoperative refractive results values 406 as a result of a previously inserted intraocular lens, and associated initial refractive power values 408 of the previously inserted intraocular lens as ground truth data.

These vectors are respectively used as training data records 403 for the ML system 410, in order to create the corresponding ML model 412. This is followed by an application 414 of the ML system in clinical day-to-day use. Subsequently, the final results of the operation can then be acquired, 416, in order thus to augment the training data volume 402 in order thus to enable a larger training data volume 418.

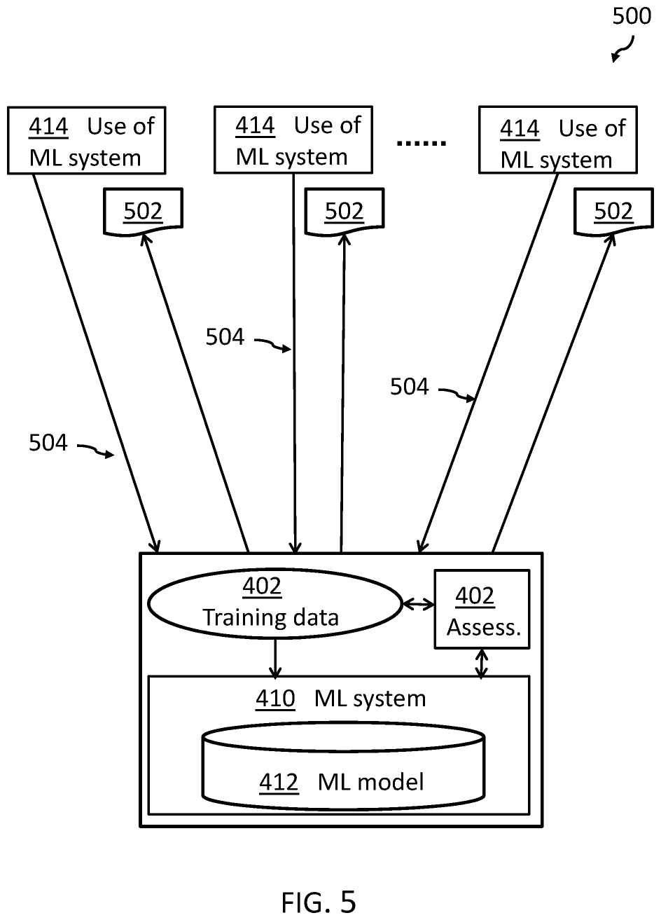

FIG. 5 shows an exemplary interaction 500 of a plurality of users with the machine learning system. In this case, a plurality of machine learning systems can be used 414, for example in different clinics. These ML systems may have the same machine learning model, at least initially.

However, in accordance with the process described in the context of FIG. 4, it is also possible to create new training data by the use of the ML system and a corresponding operation at the respective clinic, and this training data can either be processed locally or transferred to a central location, 504, for example.

In return, information may also be fed back to the entities using 414 the respective ML system. For example, this information may consist of displaying the importance indicator value on a graphical unit 502 and/or the specification about a nominal improvement and/or specification about the refractive error which can be avoided in future. Further data may also be transferred to the entities of the users 414 of the respective ML system. This could also be implemented via the amount of new training data records already transferred, with the result that it would also be possible to establish a ranking between the various clinics. Naturally, the precondition for this would be an assessment 412 of the additional training data records supplied, in the context of the already available training data 402.

It can be assumed that such a mechanism ensures that more and better training data can be collected during the use of the underlying ML system, whereby the training of the ML system 410 and the creation of a corresponding ML model 412 would be continually improved. All clinics and, in particular, the respective patients would profit from this.

The central location for retraining the ML system 410 could be at the manufacturer of the respective system or at another service provider. The data could be transferred by way of known Internet-based systems, for example using the known cloud technology methods.

It is also possible that the users 414 of the ML system do not use a local system at the clinic but instead transfer the input data to the central ML system 410 using Internet data transmission methods and are returned the respective prediction values. In this way, it would be possible to continually ensure that the ML model 412 always remains in an optimal state as a result of a centrally coordinated training.

Alternatively, the centrally trained ML system 410 can be supplied to the utilizing users, which is to say clinics, at regular intervals. Additionally, it would be possible to supply only the enlarged set of training data, with the result that non-centralized training in the clinics would be made possible. This would also have the advantage of being able to better account for clinic-specific circumstances.

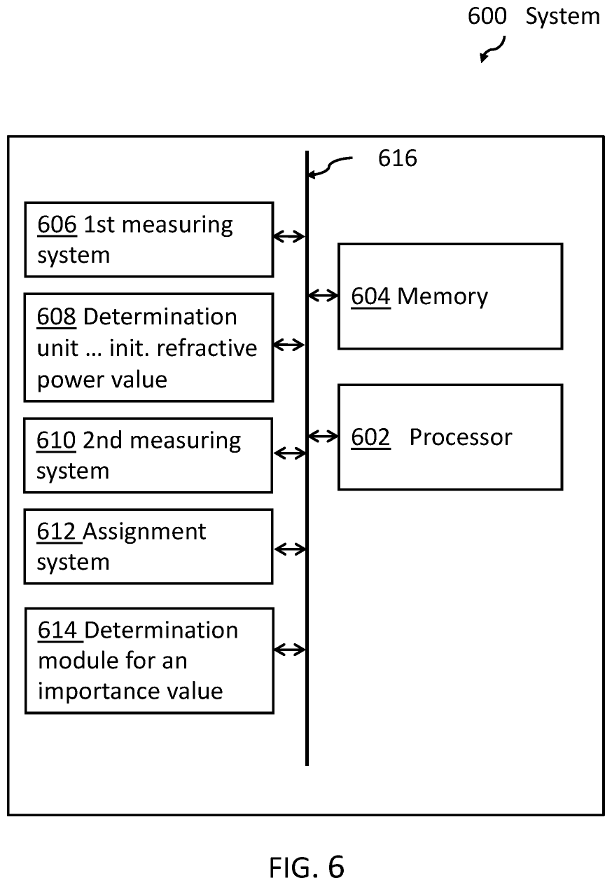

FIG. 6 shows a system 600 for increasing a training data volume for a machine learning system for determining a refractive power value for an intraocular lens to be inserted. To this end, the system 600 comprises a processor 602 and a memory 604 which is operatively connected to the processor 602 and which stores program code elements which, when executed, cause the processor 602 to measure—in particular by means of a first measuring system 606—a group of ophthalmological biometry data of a patient and determine—in particular by means of the determination unit 608 in the form of a machine learning system—an initial refractive power value for the intraocular lens to be inserted, by means of a trained machine learning system.

The corresponding machine learning system was trained previously by means of an initial training data volume consisting of tuples of previously measured ophthalmological biometry data of patients, associated postoperative refractive results values of a previously inserted intraocular lens, and associated initial refractive power values of the previously inserted intraocular lens as ground truth data for determining a corresponding machine learning model.

The measured ophthalmological biometry data and a postoperative target refraction value for the trained machine learning system are used as input data for the determination unit 608.

Furthermore, the processor 602 is caused to measure—in particular with the aid of a second measuring system 610—a postoperative refractive results value, assign—in particular using an assignment system 612—the postoperative refractive results value to the measured ophthalmological biometry data of the patient in order to form a new training data record, and determine the importance indicator value of the new training record—in particular by means of the determination module 614 for the importance value. In this case, the importance indicator value is indicative of a distance value of the new training data record in comparison with the initial training data volume.

In this case, the determination of the importance indicator value includes determining at least one element from the following group: (i) a specification regarding a nominal improvement in the future refractive power value predictions by the machine learning system, and (ii) a specification regarding a refractive error that could have been avoided if the new training data record were known prior to the determination of the initial refractive power value.

Express reference is made to the fact that the modules and units—in particular the processor 602, the memory 604, the first measuring system 606, the determination unit 608, the second measuring system 610, the assignment system 612, and the determination module 614—can be connected using electrical signal lines or via a system-internal bus system 616 for the purpose of exchanging signals or data.

In accordance with the inversion of the two above-described methods with regard to an initial refractive power and a postoperative refractive result, respectively, the next figure also describes a corresponding system which is inverted in comparison with FIG. 6.

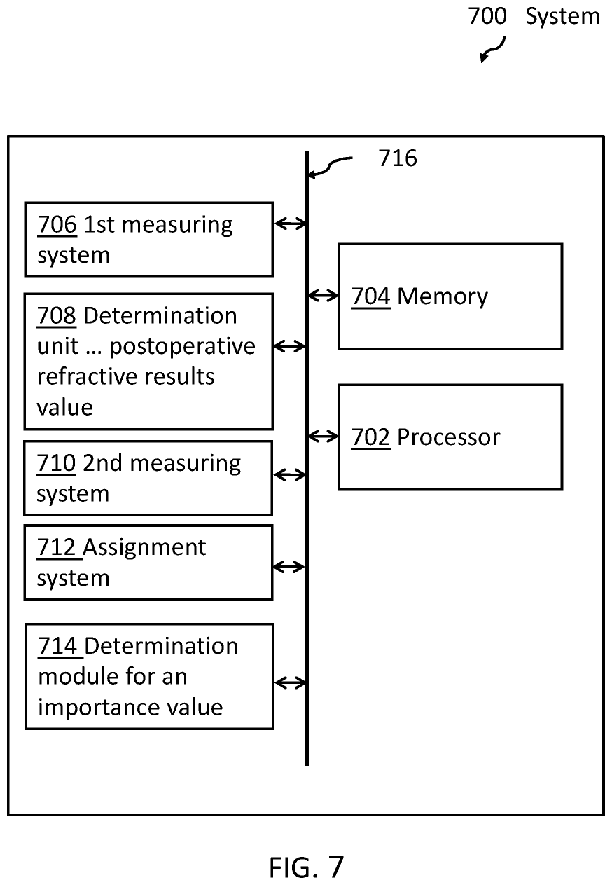

FIG. 7 shows a system 700 for increasing a training data volume for a machine learning system for determining a postoperative refractive results value of an intraocular lens to be inserted. This system comprises a processor 702 and a memory 704 which is operatively connected to the processor 702 and which stores program code elements which, when executed, cause the processor 702 to measure—in particular by means of a first measuring system 706—a group of ophthalmological biometry data of a patient and determine a postoperative refractive results value of the intraocular lens to be inserted—in particular by means of the determination unit 708 for a postoperative refractive results value—by means of a trained machine learning system.

To this end, the associated machine learning system was trained previously by means of an initial training data volume consisting of tuples of previously measured ophthalmological biometry data of patients, respectively associated initial refractive power values of a previously inserted intraocular lens, and associated postoperative refractive results values of the previously inserted intraocular lens as ground truth data for determining a corresponding machine learning model.

As input data for the prediction, the measured ophthalmological biometry data and an initial refractive power value of the inserted intraocular lens are also used here as input data for the trained machine learning system.

The processor 702 can also be caused to measure—in particular by means of the second measuring system 710—the postoperative refractive results value, assign—in particular by means of the assignment system 712—the postoperative refractive results value to the measured ophthalmological biometry data of the patient in order to form a new training data record, and determine an importance indicator value of the new training record—in particular by means of the determination module 714 for the importance value or importance indicator value. In this case, the importance indicator value is indicative of a distance value of the new training data record in comparison with the initial training data volume.

Express reference is made to the fact that the modules and units—in particular the processor 702, the memory 704, the first measuring system 706, the determination unit 708, for example in the form of a corresponding machine learning system, the second measuring system 710, the assignment system 712, and the determination module for the importance indicator value 714—can be connected using electrical signal lines or via a system-internal bus system 716 for the purpose of exchanging signals or data.

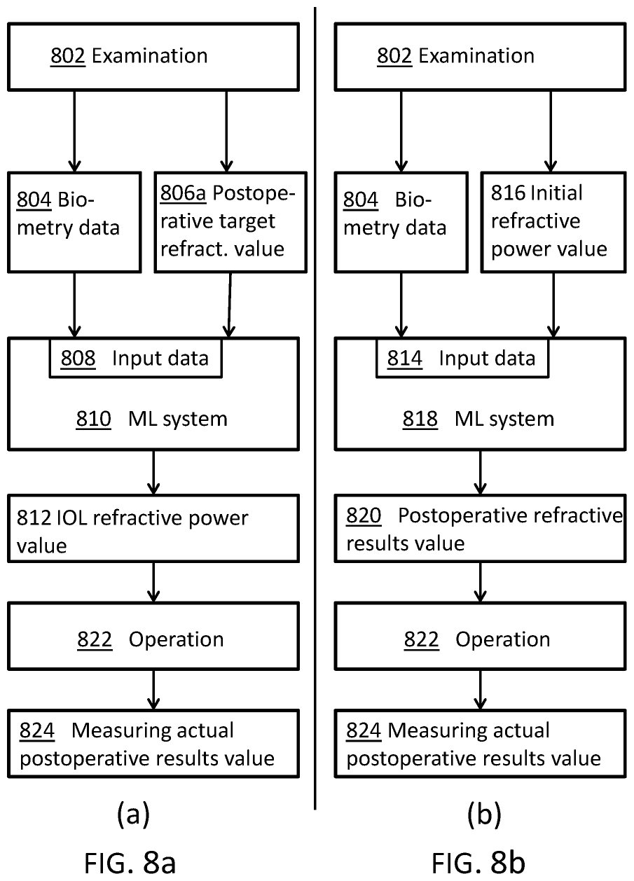

FIGS. 8a and 8b are used to elucidate the similarities of and differences between the previously presented basic methods. FIG. 8a and FIG. 8b show the two variants of the otherwise comparable methods. Both cases start with an examination 802 of the patient, in particular their eyes, or a measurement of parameter values, specifically the biometric data 804.

Then, in the case of FIG. 8A, the postoperative target refraction value 806a is made available together with the biometry data 804 as input data 808 for the trained machine learning system 810, in order to determine an initial refractive power value. This initial refractive power value is the value selected by the surgeon for the intraocular lens to be inserted before the latter is inserted. For the patient, the postoperative target refraction value is intended to be attained after the operation.

For the other case, in FIG. 8b, the biometric data 804 and an initial refractive power value 816 selected by the surgeon are made available to the trained machine learning system 818 as input data 814. Said trained machine learning system then predicts the postoperative refractive results value 820, which is to say the value expected to be obtained by the lens with the initial refractive power value 816 after the operation—in particular after the lens has grown in.

Then, the actual operation 822 of inserting the intraocular lens is implemented in both cases. In the case of FIG. 8a, a lens with the determined refractive power value 812 is inserted in the process. In the case of FIG. 8b, in turn, a lens with the initially chosen refractive power value 816 is inserted.

Subsequently, there is a measurement 824 of the actual postoperative results value of the inserted intraocular lens in the patient's eye in both cases. Thus, a check is carried out here as to whether the sought-after goal for the patient was in actual fact achieved.

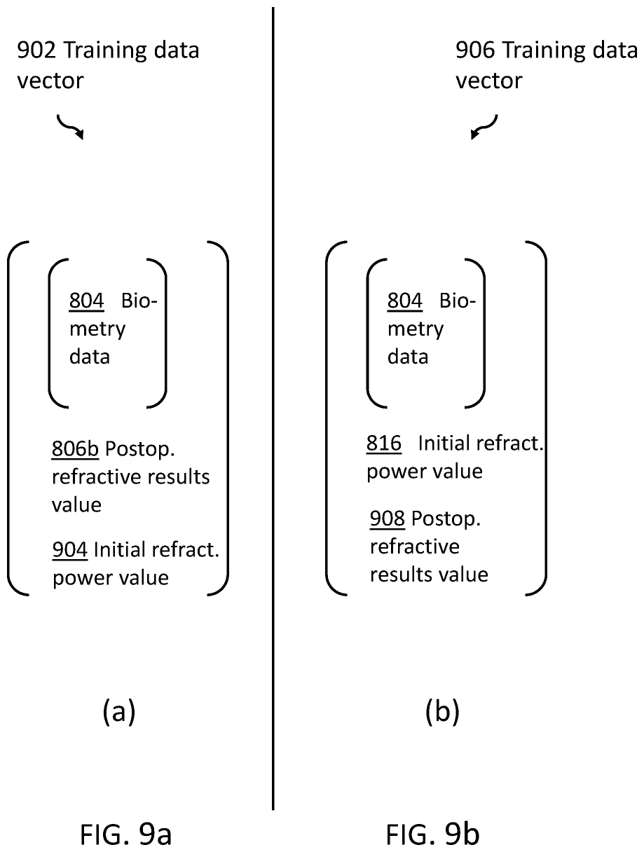

FIGS. 9a and 9b once again clarify what training data vectors are used for the two differently trained machine learning systems 810 and 818 (cf. FIGS. 8a and 8b). In the first case, the training data vector 902 consists of the biometry data 904, which in turn may be represented by a vector, the postoperative refractive results value 806b and the associated initial refractive power value 904 as ground truth value.

In the other case (case b), the training data vector 906 consists of the biometric data 804, the initial refractive power value 816, and the postoperative refractive results value 908.

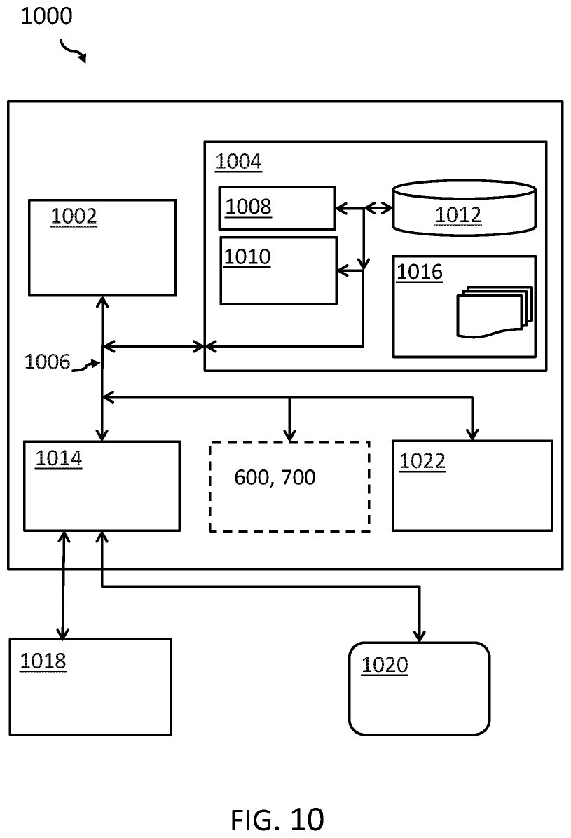

FIG. 10 shows an exemplary implementation of a computer system 1000 suitable for use together with the already presented systems.

The computer system 1000 has a plurality of general-purpose functions. The computer system may in this case be a tablet computer, a laptop/notebook computer, some other portable or mobile electronic device, a microprocessor system, a microprocessor-based system, a smartphone, a computer system with specially configured special functions, or else a constituent part of a microscope system. The computer system 1000 may be configured so as to execute computer system-executable instructions—such as for example program modules—that may be executed in order to implement functions of the concepts proposed here. For this purpose, the program modules can comprise routines, programs, objects, components, logic, data structures etc. in order to implement particular tasks or particular abstract data types.

The components of the computer system can comprise the following: one or more processors or processing units 1002, a storage system 1004 and a bus system 1006 that connects various system components, including the storage system 1004, to the processor 1002. The computer system 1000 typically comprises a plurality of volatile or non-volatile storage media accessible by the computer system 1000. The storage system 1004 may store the data and/or instructions (commands) of the storage media in volatile form—such as for example in a RAM (random access memory) 1008—in order to be executed by the processor 1002. These data and instructions realize one or more functions and/or steps of the concept presented here. Further components of the storage system 1004 can be a permanent memory (ROM) 1010 and a long-term memory 1012 in which the program modules and data (reference sign 1016) and also workflows can be stored.

The computer system comprises a number of dedicated devices (keyboard 1018, mouse/pointing device (not illustrated), screen 1020, etc.) for communication purposes. These dedicated devices can also be combined in a touch-sensitive display. An I/O controller 1014, provided separately, ensures a frictionless exchange of data with external devices. A network adapter 1022 is available for communication via a local or global network (LAN, WAN, for example via the Internet). The network adapter can be accessed by other components of the computer system 1000 via the bus system 1006. It is understood in this case, although it is not illustrated, that other devices can also be connected to the computer system 1000.

Additionally, at least parts of the system 600 or 700 for increasing a training data volume (cf. FIG. 6 or FIG. 7) may also be connected to the bus system 1006. The system 600 or the system 700 and the computer system 1000 may optionally use the memories and/or the processors and/or the bus system jointly.

The description of the various exemplary implementations of the present disclosure has been given for the purpose of improved understanding, but does not serve to directly restrict the inventive concept to these exemplary implementations. A person skilled in the art will themselves develop further modifications and variations. The terminology used here has been selected so as to best describe the basic principles of the exemplary implementations and to make them easily accessible to a person skilled in the art.

The principle presented here may be embodied as a system, as a method, combinations thereof and/or else as a computer program product. The computer program product can in this case comprise one (or more) computer-readable storage medium/media comprising computer-readable program instructions in order to cause a processor or a control system to implement various aspects of the present disclosure.

As media, electronic, magnetic, optical, electromagnetic or infrared media or semiconductor systems are used as forwarding medium; for example SSDs (solid state devices/drives as solid state memory), RAM (random access memory) and/or ROM (read-only memory), EEPROM (electrically erasable ROM) or any combination thereof. Suitable forwarding media also include propagating electromagnetic waves, electromagnetic waves in waveguides or other transmission media (for example light pulses in optical cables) or electrical signals transmitted in wires.

The computer-readable storage medium can be an embodying device that retains or stores instructions for use by an instruction executing device. The computer-readable program instructions that are described here may also be downloaded onto a corresponding computer system, for example as a (smartphone) app from a service provider via a cable-based connection or a mobile radio network.

The computer-readable program instructions for executing operations of the disclosure described here may be machine-dependent or machine-independent instructions, microcode, firmware, status-defining data or any source code or object code that is written for example in C++, Java or the like or in conventional procedural programming languages such as for example the programming language “C” or similar programming languages. The computer-readable program instructions may be executed in full by a computer system. In some exemplary implementations, there may also be electronic circuits, such as, for example, programmable logic circuits, field-programmable gate arrays (FPGAs) or programmable logic arrays (PLAs), which execute the computer-readable program instructions by using status information of the computer-readable program instructions in order to configure or to individualize the electronic circuits according to aspects of the present disclosure.

The disclosure presented here is furthermore illustrated with reference to flowcharts and/or block diagrams of methods, devices (systems) and computer program products according to exemplary implementations of the disclosure. It should be pointed out that practically any block of the flowcharts and/or block diagrams can be embodied as computer-readable program instructions.

The computer-readable program instructions can be made available to a general purpose computer, a special computer or a data processing system programmable in some other way, in order to produce a machine, such that the instructions that are executed by the processor or the computer or other programmable data processing devices generate means for implementing the functions or processes illustrated in the flowchart and/or block diagrams. These computer-readable program instructions can correspondingly also be stored on a computer-readable storage medium.

In this sense any block in the illustrated flowchart or block diagrams can represent a module, a segment or portions of instructions representing a plurality of executable instructions for implementing the specific logic function. In some exemplary implementations, the functions represented in the individual blocks can be implemented in a different order—optionally also in parallel.

The illustrated structures, materials, sequences and equivalents of all means and/or steps with associated functions in the claims hereinafter are intended to apply all structures, materials or sequences as expressed by the claims.

What is claimed is:

1. A computer-implemented method for increasing a training data volume for a machine learning system for determining an initial refractive power value for an intraocular lens to be inserted, the method comprising:
measuring a group of ophthalmological biometry data of a patient;
determining an initial refractive power value for the intraocular lens to be inserted, by a trained machine learning system which was trained by an initial training data volume consisting of tuples of previously measured ophthalmological biometry data of patients, associated postoperative refractive results values, generated by a previously inserted intraocular lens, and associated initial refractive power values of the previously inserted intraocular lens as ground truth data for determining a corresponding machine learning model, wherein the measured ophthalmological biometry data and a postoperative target refraction value are used as input data for the trained machine learning system;
measuring a postoperative refractive results value;
assigning the postoperative refractive results value to the measured ophthalmological biometry data of the patient in order to form a new training data record; and
determining an importance indicator value for the new training data record, with the importance indicator value being indicative of a distance value of the new training data record in comparison with the initial training data volume,
wherein determining the importance indicator value comprises determining at least one element from the following group:
a specification regarding a nominal improvement in the future refractive power value predictions by the machine learning system; and
a specification regarding a refractive error that could have been avoided if the new training data record were known prior to the determination of the initial refractive power value.

2. The method of claim 1, wherein the determination of the specification regarding a nominal improvement in the future refractive power predictions by the machine learning system comprises temporarily adding the new training data record to the initial training data and determining a prediction accuracy of the machine learning model.

3. The method of claim 1, further comprising increasing the importance indicator value if there are no training data within a previously defined radius around the new training data.

4. The method of claim 1, further comprising displaying, on a graphical unit, the importance indicator value and/or the specification about a nominal improvement and/or the specification about the refractive error.

5. The method of claim 1, further comprising transmitting the measured postoperative refractive results value to a training data memory.

6. The method of claim 1, further comprising retraining the machine learning system using the initial training data and the new training data record.

7. The method of claim 1, wherein the importance indicator value is determined after a number of postoperative refractive results values for one type of inserted intraocular lenses, the number being determined in advance.

8. The method of claim 1, wherein the importance indicator value for a selected type of an intraocular lens is increased by a predefined factor,
wherein the factor is increased depending on an amount of training data for the selected type of intraocular lens in comparison with the overall amount of training data, or
wherein the factor is increased if the amount of training data for the selected type of intraocular lens is less than a threshold value determined in advance.

9. The method of claim 1, wherein the importance indicator value is determined after collecting an amount of training data in the form of postoperative refractive results values for given ophthalmological biometry data and inserted intraocular lenses, the amount being determined in advance.

10. The method of claim 1, further comprising:
determining a number of measurements of the postoperative refractive results value by one entity; and
transferring the number of measurements by the entity in comparison with the number of comparable measurements by other entities.

11. A computer-implemented method for increasing a training data volume for a machine learning system for determining a postoperative refractive results value of an intraocular lens to be inserted, the method comprising:
measuring a group of ophthalmological biometry data of a patient;
determining a postoperative refractive results value of the intraocular lens to be inserted, by a trained machine learning system which was trained by an initial training data volume consisting of tuples of previously measured ophthalmological biometry data of patients, associated initial refractive power values of a previously inserted intraocular lens, and associated postoperative refractive results values, generated by the previously inserted intraocular lens, as ground truth data for determining a corresponding machine learning model, wherein the measured ophthalmological biometry data and an initial refractive power value of the inserted intraocular lens are used as input data for the trained machine learning system;
measuring the postoperative refractive results value;
assigning the postoperative refractive results value to the measured ophthalmological biometry data of the patient in order to form a new training data record; and
determining an importance indicator value for the new training data record, with the importance indicator value being indicative of a distance value of the new training data record in comparison with the initial training data volume.

12. A system for increasing a training data volume for a machine learning system for determining a refractive power value for an intraocular lens to be inserted, the system comprising:
a processor and a memory which is operatively connected to the processor and which stores program code elements which, when executed, cause the processor to:
measure a group of ophthalmological biometry data of a patient;
determine an initial refractive power value for the intraocular lens to be inserted, by a trained machine learning system which was trained by an initial training data volume consisting of tuples of previously measured ophthalmological biometry data of patients, associated postoperative refractive results values, generated by a previously inserted intraocular lens, and associated initial refractive power values of the previously inserted intraocular lens as ground truth data for determining a corresponding machine learning model, wherein the measured ophthalmological biometry data and a postoperative target refraction value are used as input data for the trained machine learning system;
measuring a postoperative refractive results value;
assigning the postoperative refractive results value to the measured ophthalmological biometry data of the patient in order to form a new training data record; and
determining an importance indicator value for the new training data record, with the importance indicator value being indicative of a distance value of the new training data record in comparison with the initial training data volume,
wherein determining the importance indicator value comprises determining at least one element from the following group:
a specification regarding a nominal improvement in the future refractive power value predictions by the machine learning system; and
a specification regarding a refractive error that could have been avoided if the new training data record were known prior to the determination of the initial refractive power value.

13. A system for increasing a training data volume for a machine learning system for determining a postoperative refractive results value of an intraocular lens to be inserted, the system comprising:
a processor and a memory which is operatively connected to the processor and which stores program code elements which, when executed, cause the processor to:
measure a group of ophthalmological biometry data of a patient;
determine a postoperative refractive results value of the intraocular lens to be inserted, by a trained machine learning system which was trained by an initial training data volume consisting of tuples of previously measured ophthalmological biometry data of patients, associated initial refractive power values of a previously inserted intraocular lens, and associated postoperative refractive results values, generated by the previously inserted intraocular lens, as ground truth data for determining a corresponding machine learning model, wherein the measured ophthalmological biometry data and an initial refractive power value of the inserted intraocular lens are used as input data for the trained machine learning system;
measuring the postoperative refractive results value;
assigning the postoperative refractive results value to the measured ophthalmological biometry data of the patient in order to form a new training data record; and
determining an importance indicator value for the new training data record, with the importance indicator value being indicative of a distance value of the new training data record in comparison with the initial training data volume.

14. A computer program product for increasing training data volume for a machine learning system for determining a refractive power value for an intraocular lens to be inserted, wherein the computer program product comprises a computer-readable storage medium having program instructions stored thereon, the program instructions being executable by one or more computers or control units and prompting the one or more computers or control units to carry out the method as per claim 1.

15. A computer program product for increasing training data volume for a machine learning system for determining a postoperative refractive results value of an intraocular lens to be inserted, wherein the computer program product comprises a computer-readable storage medium having program instructions stored thereon, the program instructions being executable by one or more computers or control units and prompting the one or more computers or control units to carry out the method as per claim 11.
